(19) United States
(12) Patent Application Publication
(10) Pub. No.: US 2005/0251096 A1
(43) Pub. Date: Nov. 10, 2005

(54) Syringe assembly with improved cap and luer connector

(76) Inventors: George Armstrong, Franklin Park, NJ (US); Ernest Balestracci, Iselin, NJ (US); Barry DeDominicis, Holland, PA (US); Gayle Heffernan, Middlesex, NJ (US); Charles Quirco, Warren, NJ (US)

Correspondence Address: BRACCO RESEARCH USA INC., 305- COLLEGE ROAD EAST, PRINCETON, NJ, 08540, US

(21) Appl. No.: 10/842,610

(22) Filed: May 10, 2004

(51) Int. Cl. A61M 5/315

(52) U.S. Cl. 604/218; 604/181

(57) ABSTRACT

A prefilled syringe assembly that includes a syringe barrel of cylindrical configuration having an inner surface that defines a cylindrical chamber, a shoulder portion connected to the cylindrical chamber, a neck portion connected to the shoulder portion and a tapered tip that terminates at the distal end. The tapered tip extends a first distance along the neck portion toward the distal end and having a shaft and a bore therethrough. The syringe barrel further includes a luer collar that terminates at the distal end and extends a second distance along the longitudinal axis from the neck portion toward the distal end such that the second distance is about equal to the first distance. The prefilled syringe assembly also includes a piston that is received through the proximal end and is positioned within the syringe barrel. The prefilled syringe assembly provides an elastomeric plug that is disposed at the distal end which protects the tapered tip from external forces and microscopic crazing during sterilization.

### FIELD OF THE INVENTION

The present invention relates to a cap and luer connector for a prefilled syringe assembly. More particularly, this invention relates to a tapered luer tip that is positioned within the distal end of the prefilled syringe assembly and is protected by the cap and luer connector.

### BACKGROUND OF THE INVENTION

One conventional type of syringe is a disposable syringe which is typically manufactured from an inexpensive thermoplastic material, such as polyethylene or the like. Such syringes may have a standard end fitting for releasably receiving a hub from which a hollow metal needle or tubing conduit projects. In particular, such syringes typically have a 6% Luer taper conical nozzle fitting with a surrounding annular collar that has a double-start, right hand, internal thread as shown in FIG. 1 of Part 2 of the International Standard ISO 594-2 (Second Edition 1998-09-01, Reference No. ISO 594-2:1998E)) as published by the International Organization for Standardization, Case postale 56, CH-1211 Geneva 20, Switzerland.

The needle or tubing to be attached to the syringe end fitting projects from a molded hub having a pair of laterally extending lugs which are adapted to threadingly engage the double-start thread in the fitting collar. The molded hub defines a tapered female configuration for receiving the tapered conical nozzle of the syringe fitting as the hub is screwed into the syringe collar. Such an assembly has long been widely used.

The syringe may be prefilled as is the case with, for example, Bracco Diagnostics model ISOVUE (Iopamidol Injection) Prefilled Injector Syringe with Pressure Tubing. The prefilled syringe barrel is typically distributed without the needle attached. Instead, the prefilled syringe is supplied with a cap or cover over the projecting nozzle. The projecting nozzle is at risk for damage due to handling because it extends beyond the threaded fitting collar. Therefore, any pressure or forces that are applied to cap during handling will be indirectly applied to tapered tip. Upon removal of the cap, the handling and connecting forces (e.g. forces associated with attachment of a tubing conduit, etc.) will be applied directly to the tapered tip. Such pressures or forces can cause the tapered tip to crack and sever.

Further, it is desirable to provide such a prefilled syringe to the ultimate user with the fluid product contained in the syringe in a sterile condition. Therefore, as part of the manufacturing process, it is common for such syringes to undergo a terminal sterilization process prior to final packaging. Very high temperatures can be used in such sterilization processes. It is known in the art that plastics can be vulnerable to degradation and microscopic crazing when exposed to high temperatures or other sterilization processes. Consequently, it is common for such plastics to experience microscopic crazing after such processes.

The present invention provides a syringe assembly with an improved cap and tapered luer tip that reduces the risk of damage to the projecting nozzle during handling and minimizes degradation and microscopic crazing of the nozzle during a sterilization process.

### SUMMARY OF THE INVENTION

In accordance with the present invention there is provided a prefilled syringe assembly equipped with a syringe barrel and a piston.

The syringe barrel is of cylindrical configuration and has a longitudinal axis that extends between a distal end and a proximal end. The syringe barrel includes an inner surface that defines a cylindrical chamber having a medical fluid therein. The syringe barrel further includes a shoulder portion that is connected to the cylindrical chamber and a neck portion that is connected to the shoulder portion. The syringe barrel also includes a tapered tip that terminates at the distal end, wherein the tapered tip extends a first distance along the longitudinal axis from the neck portion toward the distal end. The tapered tip includes a shaft and a bore therethrough. The syringe barrel additionally includes a luer collar that terminates at the distal end. The luer collar extends a second distance along the longitudinal axis from the neck portion toward the distal end such that the second distance is about equal to the first distance.

The first distance may be no less than 3 mm less than the second distance and the second distance may be no more than 4 mm more than the first distance.

The piston is received through the proximal end and is positioned within the syringe barrel. The piston adapted for movement along the longitudinal axis.

The syringe barrel is made of a material selected from the group consisting of polyolefin polymers, polyolefin copolymers, polypropylene, olefin polymers, olefin copolymers, cyclic olefins, polyester or methylpentene.

The luer collar further provides a threaded connection to which an injection needle or a connector equipped with a tubing conduit can be attached.

The prefilled syringe assembly may further include an elastomeric plug of cylindrical configuration, wherein the elastomeric plug disposed at the distal end. The plug may be made from a plastic, an elastomeric compound or a combination of plastic and elastomeric compound.

The prefilled syringe assembly may be used as a hand operated device or in conjunction with a semi-automatic injection device or in conjunction with an automatic injection device.

The prefilled syringe assembly may optionally include a means of releasably engaging an injection device.

The prefilled syringe assembly may optionally include a locking mechanism disposed on the syringe barrel for interfacing with an injection device.

The prefilled syringe assembly may also include at least one locking tab disposed at the proximal end of the syringe barrel for interfacing with an injection device. The at least one locking tab includes a first pair of locking tabs that are positioned 180° opposite a second pair of locking tabs that are disposed on an outer surface of the syringe barrel and about the longitudinal axis. a third locking tab positioned 180° opposite a fourth locking tab disposed on the outer surface of the syringe barrel and about the longitudinal axis, wherein the first and second pairs of locking tabs are 90° offset from the third and fourth locking tabs.

The prefilled syringe assembly of this embodiment may also include a flat ring that extends away from the longitudinal axis and is disposed at the proximal end of the syringe barrel. The flat ring serves as a mechanical locating device for positioning the syringe assembly at a filling station.

In another embodiment of the present invention the tapered tip of the prefilled syringe assembly may include a radial reinforcing shoulder that extends about the tapered tip. The radial shoulder is concentric with the longitudinal axis.

In another embodiment of the present invention the tapered tip of the prefilled syringe assembly may include a plurality of radial reinforcing ribs that extend longitudinally from the base of the luer collar and along the shaft of the tapered tip toward the distal end. The plurality of radial reinforcing ribs extend longitudinally from the base of the luer collar and along the shaft of the tapered tip toward the distal end a predetermined distance of at least 3 mm and not more than 5 mm.

In a preferred embodiment the radial reinforcing ribs extend longitudinally from the base of the luer collar and along the shaft of the tapered tip toward the distal end a predetermined distance of 4 mm. The plurality of radial reinforcing ribs may provide at least 3 radial reinforcing ribs. In this embodiment, the thickness of the radial reinforcing ribs are at least 1 mm.

In another embodiment the prefilled syringe assembly may include an elastomeric plug of cylindrical configuration. The elastomeric plug is disposed at the distal end and includes a widened top portion that facilitates its removal. The elastomeric plug further includes an outer skirt that is connected to the widened top portion which is contiguous with the outer wall of the luer collar. The elastomeric plug also includes an inner skirt that is connected to the widened top portion which is contiguous with the shaft of the tapered tip.

The elastomeric plug minimizes microscopic crazing of the tapered tip and/or luer collar during steam sterilization and protects the tapered tip and luer collar from breakage during handling.

The elastomeric plug is made of a material selected from the group consisting of natural rubber, butyl or halobutyl rubber.

The elastomeric plug may be made from a plastic, an elastomeric compound or a combination of plastic and elastomeric compound.

The prefilled syringe assembly may further include a means of releasably engaging an injection device;

The prefilled syringe assembly may also include a locking mechanism that is disposed on the syringe barrel for interfacing with an injection device;

The prefilled syringe assembly may additionally include at least one locking tab disposed at the proximal end of the syringe barrel for interfacing with an injection device. The at least one locking tab have a first pair of locking tabs that are positioned 180° opposite a second pair of locking tabs that are disposed on an outer surface of the syringe barrel and about the longitudinal axis. This embodiment may have a third locking tab that is positioned 180° opposite a fourth locking tab that is also disposed on the outer surface of the syringe barrel and about the longitudinal axis. The first and second pairs of locking tabs are 90° offset from the third and fourth locking tabs.

The prefilled syringe assembly may optionally provide a flat ring that extends away from the longitudinal axis and is disposed at the proximal end of the syringe barrel for preventing medical fluid from dripping into the injection device and serving as a mechanical locating device for positioning the syringe assembly at a filling station.

### BRIEF DESCRIPTION OF THE DRAWINGS

FIG. 1 is a perspective view of a prior art syringe barrel assembly;

FIG. 2 is a cross-sectional view of a distal end of the prior art syringe barrel and cap assembly;

FIG. 3 is a perspective view of a syringe barrel assembly of the present invention;

FIG. 4 is a cross-sectional view of a distal end of the syringe barrel of the present invention;

FIG. 5 is a second cross-sectional view of the distal end of the syringe barrel and cap of the present invention;

FIG. 6 is a cross-sectional view of another embodiment showing a distal end of a syringe barrel and cap of the present invention;

FIG. 7 is a partially broken away perspective view of another embodiment of the present invention showing a tapered tip with a radial reinforcing shoulder extending about the tapered tip.

FIG. 8 is a partially broken away perspective view of another embodiment of the present invention showing the tapered tip and radial reinforcing ribs;

FIG. 9 is a cross-sectional view of the tapered tip, radial reinforcing ribs and luer connector taken along line 8-8 of FIG. 7;

### DETAILED DESCRIPTION OF THE INVENTION

While the invention includes embodiments in many different forms, there is shown in the drawings and will herein be described in detail preferred embodiments of the invention with the understanding that the present disclosure is to be considered as exemplary of the principles of the invention and is not intended to limit the invention to the embodiments illustrated.

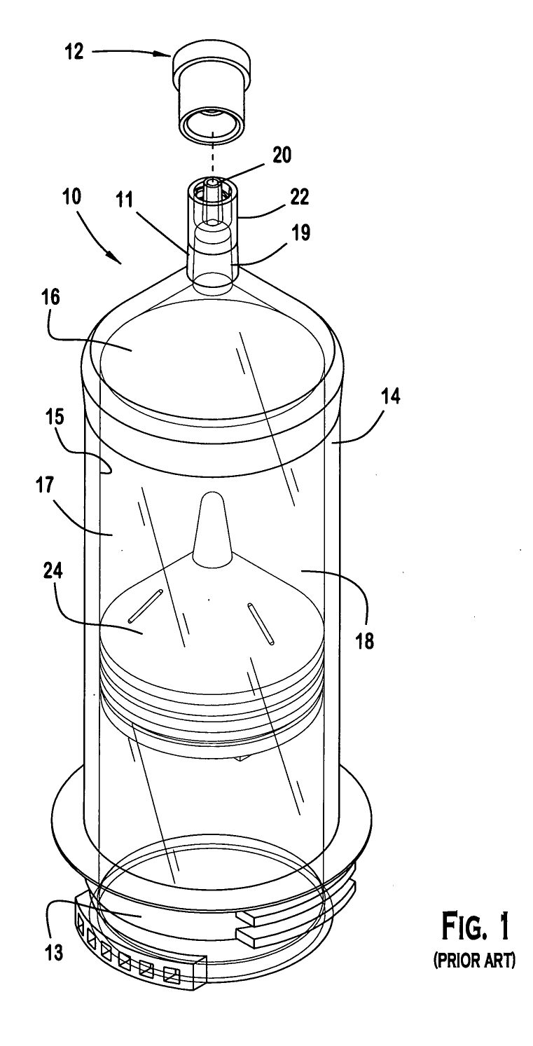

Referring to FIG. 1, there is shown in perspective view a prior art syringe assembly. Syringe assembly 10 comprises cap 12, syringe barrel 14, piston 24. Syringe barrel 14 having a distal end 11 and a proximal end 13 includes an inner surface 15 that defines a cylindrical chamber 18 having a medical fluid 17 therein. An injection device (not shown) may be used in conjunction with the syringe assembly 10 to inject the medical fluid 17 into a site (not shown).

For purposes of the description of the prior art and present invention, the term “distal end” refers to the end furthest from the end which receives the piston, whereas the term “proximal end” refers to the end which receives the piston.

Syringe barrel 14 further includes a shoulder 16 that is connected to the cylindrical chamber 18, a neck 19 that is connected to the shoulder 16, a tapered luer tip 20 that terminates at the distal end 11, and a Luer lock collar 22 that terminates at the distal end. The tapered tip 20 extends beyond the Luer lock collar 22 by about 2.1 mm to a furthest point 21 of the distal end 11. The tapered tip 20 and Luer lock collar 22 are designed to international standard (ISO 594-2) entitled, “Conical fittings with 6% (Luer) taper for syringes, needles and certain other medical equipment”, which specifies, among other things, that the tip extends 2.1 mm beyond the Luer collar. Thus, design of the tapered tip 20 and Luer lock collar 22 in prior art syringes does not deviate from the ISO standard.

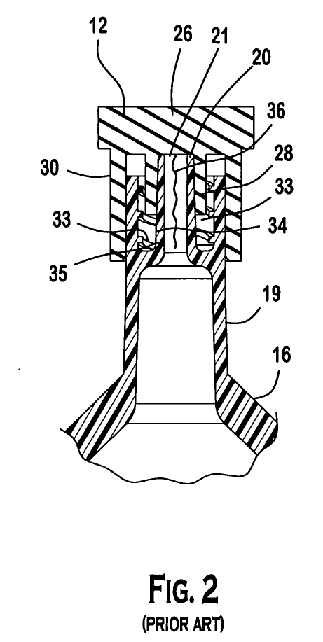

Referring to FIG. 2, there is shown a cross-sectional view of the distal end 11 of the prior art syringe barrel 14 and cap 10. The cap 12 includes a widened top 26 connected to an inner skirt 28 and an outer skirt 30. Since the tapered tip 20 extends beyond the Luer lock collar 22, cap 12 rests directly on tapered tip 20. Therefore, any pressure or forces that are applied to cap 12 during handling will be indirectly applied to tapered tip 20. Upon removal of the cap 12, the handling and connecting forces (e.g. forces from connecting tubing etc.) will be applied directly to the tapered tip 20. Such pressures or forces can cause the tapered tip 20 to crack and sever at break points 34 and 36.

In addition, the inner skirt 28 does not cover the entire tapered tip 20. Since the inner skirt 28 does not cover the entire tapered tip 20, gaps 33 are formed. This leaves a lower portion 35 of the tapered tip 20 that is not covered by inner skirt 28. Since the inner skirt 28 does not cover the lower portion 35 of the tapered tip 20, the lower portion 35 is vulnerable to microscopic crazing during sterilization, i.e. steam sterilization, or any other method of sterilization. It will be recognized by those skilled in the art that microscopic crazing can visibly cloud and effectively weaken an area that has been affected.

The present invention is directed to a syringe assembly with an improved cap and luer connector design that reduces the risk of damage during handling and minimizes microscopic crazing of the tapered tip (particularly the lower portion of the tapered tip) during sterilization. This and other advantageous features will be now explained in the description of the invention in reference to the drawings.

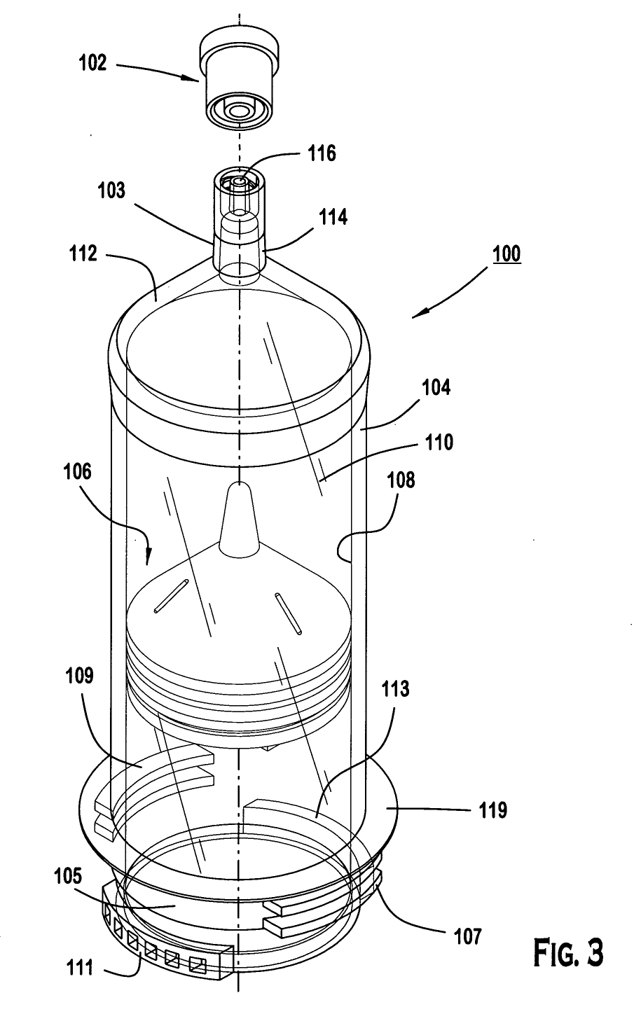

Referring to FIG. 3, there is shown a perspective view of the syringe assembly of the present invention. Syringe assembly 100 is designed for injecting a medical fluid into a site (not shown) and comprises a syringe barrel 104, a piston 106 and optionally a cap 102.

Syringe barrel 104 is of cylindrical configuration and has a longitudinal axis that extends between a distal end 103 and a proximal end 105. Syringe barrel 104 includes an inner surface 108 that defines a cylindrical chamber 110. Cylindrical chamber 110 houses the medical fluid to be injected.

Syringe barrel 104 includes a shoulder portion 112 that is connected to the cylindrical chamber 110 and a neck portion 114 that is connected to the shoulder portion 112. The piston 106 is received through the proximal end 105 and is positioned within the syringe barrel 104. Piston 106 is adapted for movement along the longitudinal axis. The materials used to make the piston 106 are conventional and known to those skilled in the art.

In a preferred embodiment, the piston may be made of one, two or more pieces. The piston may, for example, be a single piece component, or a two piece component consisting of a core and a flexible cover piece attached to or fitting over onto the core (e.g., allowing the piston to seal the barrel of the syringe). In the latter case, the core is preferably made of a relatively hard plastic such as a polyolefin (e.g., polypropylene or polycarbonate), and the flexible rubber piece is preferably made of a flexible rubber elastomer, such as natural rubber, butyl or halobutyl rubber; the two pieces may be pre-assembled to form the piston prior to insertion into the barrel.

In another embodiment, the syringe 100 may further comprise a means of releasably engaging an injection device. Such means are known to those skilled in the art and may include, for example, a locking mechanism disposed on the syringe barrel. For example, in the case of a front loading device syringe barrel 104 may optionally include at least one locking tab that is disposed at the proximal end 105 of the syringe barrel 104 for interfacing with an injection device. The injection device may be hand held, semi-automatic or automatic.

In another embodiment, the at least one locking tab may comprise a first pair of locking tabs 107 that are positioned about 180° opposite a second pair of locking tabs 109. Both the first pair and second pair of locking tabs 107, 109 are disposed on an outer surface of the syringe barrel 104 and about the longitudinal axis. This embodiment may optionally further include a third locking tab 111 that is positioned about 180° opposite a fourth locking tab 113. Both the third locking tab 111 and fourth locking tab 113 are disposed on the outer surface of the syringe barrel 104 and about the longitudinal axis. The first and second pairs of locking tabs 107, 109 are 90° offset from the third and fourth locking tabs 111, 113.

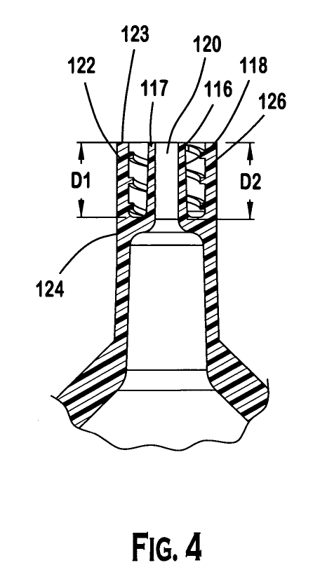

Referring now to FIG. 4, there is shown a cross-sectional view of the distal end 103 of the syringe barrel 104 of the present invention. Syringe barrel 104 further includes a tapered tip 116 that terminates at a first outer end 117 of the distal end 103. The tapered tip 116 extends a first distance D₁ along the longitudinal axis from the neck portion 114 toward the distal end 103. The tapered tip 116 has a shaft 118 and a bore therethrough 120.

Syringe barrel 104 provides a luer collar 122 that terminates at a second outer end 123 of the distal end 103. Luer collar 122 extends a second distance D₂ along the longitudinal axis from the neck portion 114 toward second outer end 123 of the distal end 103 such that the second distance D₂ is about equal to the first distance D₁. The luer collar 122 includes a base portion 124 that connects the luer collar 122 to the tapered tip 116 and an outer wall 126 that connects the base portion 124 and the neck portion 114.

Syringe barrel 104 may be made of any suitable plastic, and is preferably made of polyolefin, including polyolefin polymers, polyolefin copolymers and blends, especially polypropylene or blends thereof with polyethylene, or olefin polymers and copolymers, including methylpentene or the like polyolefins, and cyclic olefins or polyester.

Syringe barrel 104 may optionally also include a flat ring 119 that extends away from the longitudinal axis and is disposed at the proximal end 105 of the syringe barrel 104. Flat ring 119 serves as a mechanical locating device for positioning the syringe assembly at a filling station (not shown). In addition, flat ring 119 may serve to prevent the medical fluid from flowing into the injection device during use. Flat ring 119 may be made from any suitable plastic, and is preferably made from the same family of materials that were disclosed above in connection with the syringe barrel 104.

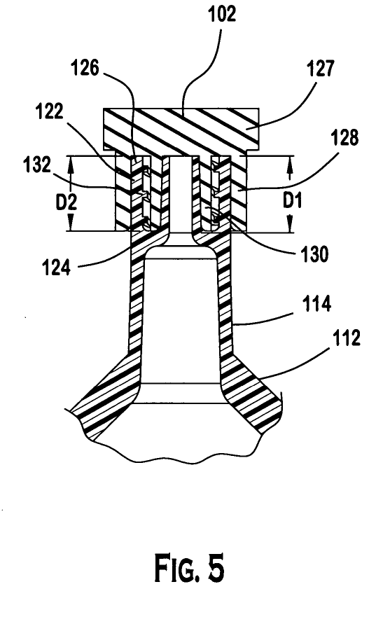

Referring to FIG. 5, there is shown a cross-sectional view of the elastomeric plug or cap 102 and distal end 103 of the syringe barrel 104. The elastomeric plug 102 is of cylindrical configuration and includes a widened top portion 127. The widened top portion 127 enables one to grasp and remove the elastomeric plug 102 from the syringe assembly 100. The elastomeric plug 102 further includes an outer skirt 128 that is connected to the widened top portion 127. The outer skirt 128 is contiguous with the luer collar 122 along the second distance D₂. The elastomeric plug 102 also includes an inner skirt 130 that is connected to the widened top portion 127. The inner skirt 130 is contiguous with the tapered tip 116 along the first distance D₁.

The luer collar 122 may optionally provide a threaded connection 132 to which an injection needle or a luer connector equipped with a tubing conduit (not shown) can be attached.

Some of the plastics that are molded into syringe barrels can exhibit hazing or microcrazing or microcracking after being exposed to conditions of sterilization. The hazing or microcrazing or microcracking can be a result of a change in the crystalline structure of the plastic during the sterilization process. High temperature, high pressure and/or steam exposure, as can be associated with sterilization, or other sterilization processes can potentially decrease the strength characteristics of plastic. Plastic hazing is minimized in the location where there is contact with a rubber elastomeric compound. The elastomer provides protection to the plastic area it contacts. The effect of the elastomer contact is visually noticeable after sterilization since the plastic will exhibit less haze in the contact area than in the noncontact areas. Applicants have found that microscopic examination of the plastic also demonstrates there is less microcracking in the plastic areas which contact a rubber elastomer during sterilization.

With respect to the present invention, since the inner skirt 130 covers and is contiguous with the tapered tip 116 and the outer skirt 128 covers and is contiguous with the luer collar 122, the elastomeric plug 102 can effectively be utilized as a shield in the manner described above to protect the tapered tip 116 and luer collar 122 from microcrazing and to avoid a decrease in plastic strength during steam sterilization.

The elastomeric plug 102 may be made of any suitable plastic or combination of plastic and elastomer, and is preferably made of a flexible rubber elastomer such as natural rubber, butyl or halobutyl rubber or blends thereof.

As previously disclosed, the distance D₁ between the neck 114 and the first outer end 117 of the tapered tip 116, and the neck 114 and the second outer end 123 of the luer collar 122 are about equal. Since D₁ and D₂ are about equal, the elastomeric plug rests on the first outer end 117 of the shortened tapered tip 116, and the second outer end 123 of the luer collar 122.

Therefore, elastomeric plug 102 no longer directly rests on the tapered tip 116 alone (as is with the case with the prior art). The ratio of areas between the second outer end 123 of the luer collar 122 and the first outer end of the tapered tip 116 of the present invention is approximately 5:1. Thus, virtually all of the pressure applied to the elastomeric plug 102 during handling will be absorbed by the second outer end 123 of the luer collar 122. Accordingly, it is at the heart of the present invention that the shortened tapered tip 116 and elastomeric plug 102 design of the present invention inherently protects the tapered tip 116 from the vulnerability of breakage during handling.

In another embodiment of the invention, the first distance D₁ may be provided being slightly less than the second distance D₂. However, the first distance D₁ may be no less than about 3 mm less than the second distance D₂. If so, a luer connector (not shown) that is designed to the ISO standard may not seal correctly with the tapered tip 116.

In a variation of this embodiment, the outer skirt 128 of the elastomeric plug 102 may extend beyond the outer wall 126 of the luer collar 122 into the neck portion 114 a predetermined distance of at least about 1 mm and not more than about 10 mm. Also, the inner skirt 130 of the elastomeric plug 102 does not reach the base portion 124. The inner skirt 130 may extend from the widened top portion 127 toward the proximal end 105 a predetermined distance of about 6 mm, but not more than about 10 mm.

In a further variation of this embodiment, the outer skirt 128 of the elastomeric plug 102 is not provided. In this variation, the widened top portion 127 may be provided narrower such that it is about the same diameter as the inner skirt 130. As was the case with the previous variation, the inner skirt 130 of the elastomeric plug 102 does not reach the base portion 124 and the inner skirt 130 may extend from the narrowed top portion toward the proximal end 105 a predetermined distance of about 6 mm, but not more than about 10 mm.

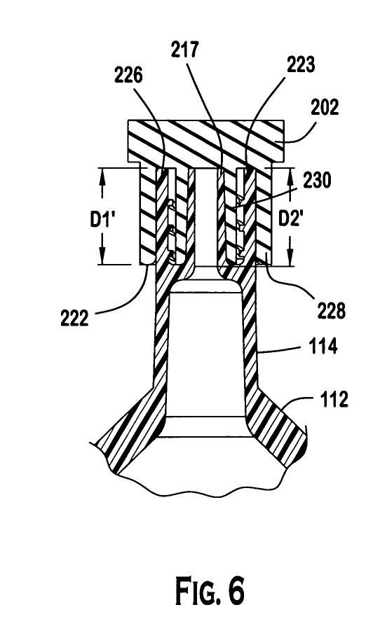

Referring to FIG. 6, there is shown in cross-sectional view of an alternate embodiment of the distal end of the present invention. In this embodiment, all of the features of the syringe assembly of the present invention as described above are the same except tapered tip 116 is not shortened. Rather, second outer end 223 of outer wall 226 of luer collar 222 is provided elongated such that the first and second distances D₁′ and D₂′ are still generally equal. In addition, since the second outer end 223 of outer wall 226 of the luer collar 222 has been elongated, inner skirt 230 is also elongated and is contiguous with the tapered tip 216 as shown. Accordingly, the present embodiment inherently protects the tapered tip 216 from the vulnerability of breakage during handling as well.

In a variation of this embodiment of the invention, second distance D₂ may be slightly greater than the first distance D₁. However, the second distance D2 may be no more than about 4 mm greater than the first distance D₁. If so, a luer connector (not shown) that is designed to the ISO standard may not seal correctly with the tapered tip 216.

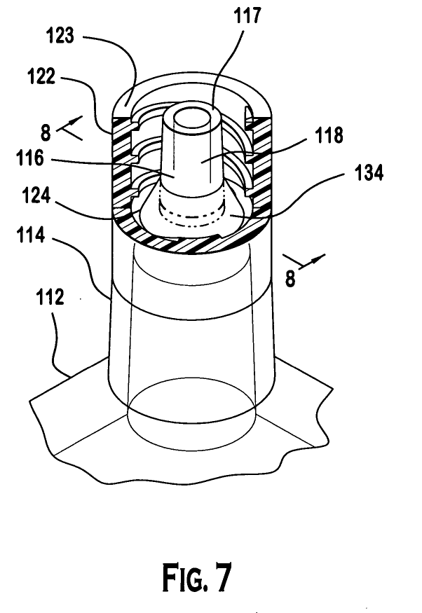

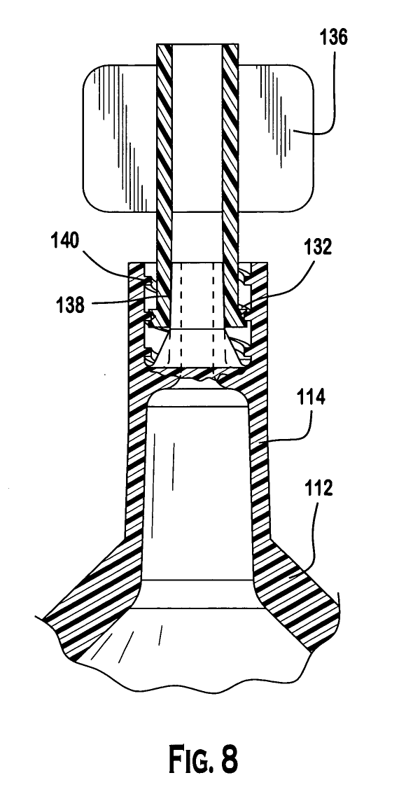

Referring to FIGS. 7-8, there is shown a partially broken away perspective view and a cross-sectional view taken along line 8-8 of FIG. 7 of another embodiment of the present invention. In this embodiment, all of the features of the syringe assembly of the present invention as described above are the same except a radial shoulder 134 is provided. The radial shoulder 134 extends about the tapered tip 116 and is concentric with the longitudinal axis. The base portion 124 connects the luer collar 122 to the radial shoulder 134. The radial shoulder 134 provides added strength to the base portion 124 of the tapered tip 116. The radial shoulder 134 acts to enhance the resiliency of the tapered tip 116 of the syringe barrel 104 and thus further reduces the chance of breakage during handling.

The radial shoulder 134 may be molded as a single component of the syringe barrel 104 or may be fabricated separately and bonded to the syringe barrel. It will be recognized by those skilled in the art that there are numerous ways to bond one plastic to another. Those methods include, but are not limited to, heat welding, sonic welding and the use of adhesives or epoxies.

FIG. 8 shows a typical luer connector 136 connected to the luer collar 122 and tapered tip 116 of the present embodiment. The luer connector 136 is screwed into threaded connection 132 of the luer collar 122. An inner portion 140 of the luer connector 136 seals against the tapered tip 116 at an area of contact 138. Upon assembly, the luer connector 136 bottoms out at the area of contact 138 on the shaft 118 of the tapered tip 116 before any part of the luer connector 136 can hit radial shoulder 134.

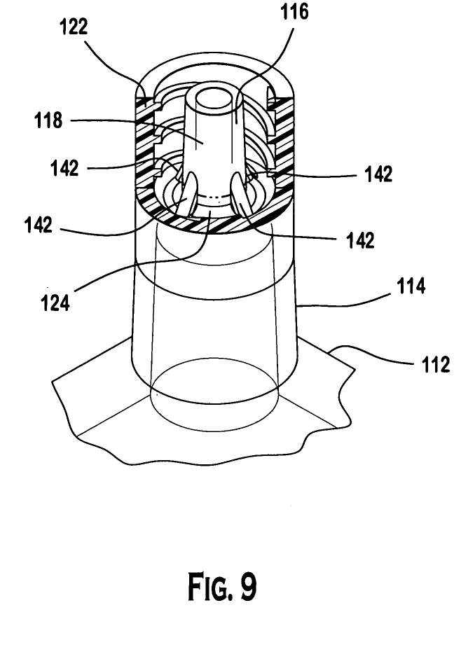

FIG. 9 shows a partially broken away perspective view of another embodiment of the present invention. In this embodiment, all of the features of the syringe assembly of the present invention as described above with respect to FIGS. 7-8 are the same except for the following. Instead of providing a single radial shoulder 134, a plurality of radial reinforcing ribs 142 that extend longitudinally from the base portion 124 of the luer collar 122 and along the shaft 118 of the tapered tip 116 are provided. Like the radial shoulder 134, the plurality of radial reinforcing ribs 142 may be molded as a single component of the syringe barrel 104 or may be fabricated separately and bonded to the syringe barrel.

The plurality of radial reinforcing ribs 142 extend toward the distal end 103 a predetermined distance. The predetermined distance ranges between at least about 2 mm and not more than about 5 mm. The ideal predetermined distance is about 4 mm. The plurality of radial reinforcing ribs 142 may further comprise at least 3 radial reinforcing ribs. In a further embodiment the plurality of radial reinforcing ribs may provide 4 radial reinforcing ribs 142. The thickness of the radial reinforcing ribs 142 are at least about 1 mm and not more than about 2 mm. An ideal thickness of the radial reinforcing ribs 142 is 1.5 mm. Further, the radial reinforcing ribs 142 range in width from about 1-4 mm.

As was the case with the single radial shoulder 134 above, the plurality of radial reinforcing ribs 142 provide added strength to the base portion 124 of the tapered tip 116. Accordingly, this design of the present invention acts to enhance the resiliency of the tapered tip 116 and thus further reduces the chance of breakage during handling as well.

Both the plurality of radial reinforcing ribs 142 and the single radial shoulder 134 may be made from any suitable plastic, and are preferably made from the same family of materials that were previously disclosed above in connection with the syringe barrel 104.

PART LISTS

| Prior art: | |
|---|---|
| Syringe assembly | 10 |
| Distal end | 11 |
| Cap | 12 |
| Proximal end | 13 |
| Syringe barrel | 14 |
| Inner surface | 15 |
| Shoulder | 16 |
| Medical fluid | 17 |
| Cylindrical chamber | 18 |
| Neck | 19 |
| Tapered tip | 20 |
| Furthest most point | 21 |
| Luer collar | 22 |
| Piston | 24 |
| Widened top | 26 |
| Inner skirt | 28 |
| Outer skirt | 30 |
| Gaps | 33 |
| Break point | 34 |
| Lower portion of tapered tip | 35 |
| Present Invention: | |
| Longitudinal axis | |
| First distance | D₁,D₁' |
| Second distance | D₂,D₂' |
| Syringe assembly | 100 |
| Elastomeric plug | 102 |
| Distal end | 103 |
| Syringe barrel | 104 |
| Proximal end | 105 |
| Piston | 106 |
| First pair of locking tabs | 107 |
| Inner surface | 108 |
| Second pair of locking tabs | 109 |
| Cylindrical chamber | 110 |
| Third locking tab | 111 |
| Shoulder portion | 112 |
| Fourth locking tab | 113 |
| Neck portion | 114 |
| Tapered tip | 116 |
| First outer end (Tip) | 117 |
| Shaft | 118 |
| Flat ring | 119 |
| Bore | 120 |
| Luer collar | 122 |
| Second outer end (Luer collar) | 123 |
| Base portion | 124 |
| Outer wall | 126 |
| Widened top portion | 127 |
| Outer skirt | 128 |
| Inner skirt | 130 |
| Threaded connection | 132 |
| Radial shoulder | 134 |
| Luer connector | 136 |
| Area of contact | 138 |
| Inner Portion | 140 |
| Plurality of reinforcing ribs | 142 |

Various modifications of the present invention disclosed will become apparent. This invention is intended to include such modifications to be limited only by the scope of the claims.

1). A prefilled syringe assembly comprising:
(A) a syringe barrel of cylindrical configuration having a longitudinal axis extending between a distal end and a proximal end, the syringe barrel including:
(1) an inner surface defining a cylindrical chamber having said medical fluid therein;
(2) a shoulder portion connected to the cylindrical chamber;
(3) a neck portion connected to the shoulder portion;
(4) a tapered tip terminating at the distal end, the tapered tip extending a first distance along the longitudinal axis from the neck portion toward the distal end and having:
(a) a shaft; and
(b) a bore therethrough; and
(5) a luer collar terminating at the distal end and extending a second distance along the longitudinal axis from the neck portion toward the distal end such that the second distance is about equal to the first distance; and
(B) a piston received through the proximal end and positioned within the syringe barrel, the piston adapted for movement along the longitudinal axis.

2). The prefilled syringe assembly of claim 1 wherein the syringe barrel is made of a material selected from the group consisting of polyolefin polymers, polyolefin copolymers, polypropylene, olefin polymers, olefin copolymers, cyclic olefins, polyester or methylpentene.

3). The prefilled syringe assembly of claim 1 wherein the luer collar further provides a threaded connection to which an injection needle or a connector equipped with a tubing conduit can be attached.

4). The prefilled syringe assembly of claim 1 wherein the first distance may be no less than 3 mm less than the second distance.

5). The prefilled syringe assembly of claim 1 wherein the second distance may be no more than 4 mm more than the first distance.

6). The prefilled syringe assembly of claim 1 further comprising an elastomeric plug of cylindrical configuration, the elastomeric plug disposed at the distal end.

7). The prefilled syringe assembly of claim 6 wherein the plug may be made from a plastic, an elastomeric compound or a combination of plastic and elastomeric compound.

8). A prefilled syringe assembly comprising:
(A) a syringe barrel of cylindrical configuration having a longitudinal axis extending between a distal end and a proximal end, the syringe barrel including:
(1) an inner surface defining a cylindrical chamber having said medical fluid therein;
(2) a shoulder portion connected to the cylindrical chamber;
(3) a neck portion connected to the shoulder portion;
(4) a tapered tip terminating at the distal end, the tapered tip extending a first distance along the longitudinal axis from the neck portion toward the distal end and having:
(a) a shaft;
(b) a bore therethrough; and
(c) a radial reinforcing shoulder extending about the tapered tip and concentric with the longitudinal axis; and
(5) a luer collar terminating at the distal end and extending a second distance along the longitudinal axis from the neck portion toward the distal end such that the second distance is generally equal to the first distance; and
(B) a piston received through the proximal end and positioned within the syringe barrel, the piston adapted for movement along the longitudinal axis.

9). The prefilled syringe assembly of claim 8 further comprising an elastomeric plug of cylindrical configuration, the elastomeric plug disposed at the distal end.

10). The prefilled syringe assembly of claim 9 wherein the plug may be made from a plastic, an elastomeric compound or a combination of plastic and elastomeric compound.

11). A prefilled syringe assembly comprising:
(A) a syringe barrel of cylindrical configuration having a longitudinal axis extending between a distal end and a proximal end, the syringe barrel including:
(1) an inner surface defining a cylindrical chamber having said medical fluid therein;
(2) a shoulder portion connected to the cylindrical chamber;
(3) a neck portion connected to the shoulder portion;
(4) a tapered tip terminating at the distal end, the tapered tip extending a first distance along the longitudinal axis from the neck portion toward the distal end and having:
(a) a shaft;
(b) a bore therethrough; and
(c) a plurality of radial reinforcing ribs extending longitudinally from the base of the luer collar and along the shaft of the tapered tip toward the distal end; and
(5) a luer collar terminating at the distal end and extending a second distance along the longitudinal axis from the neck portion toward the distal end such that the second distance is generally equal to the first distance; and
(B) a piston received through the proximal end and positioned within the syringe barrel, the piston adapted for movement along the longitudinal axis.

12). The prefilled syringe assembly of claim 11 wherein the plurality of radial reinforcing ribs extend longitudinally from the base of the luer collar and along the shaft of the tapered tip toward the distal end a predetermined distance of at least 3 mm and not more than 5 mm.

13). The prefilled syringe assembly of claim 12 wherein the radial reinforcing ribs extend longitudinally from the base of the luer collar and along the shaft of the tapered tip toward the distal end a predetermined distance of 4 mm.

14). The prefilled syringe assembly of claim 11 wherein the plurality of radial reinforcing ribs further comprises at least 3 radial reinforcing ribs.

15). The prefilled syringe assembly of claim 11 wherein the thickness of the radial reinforcing ribs are at least 1 mm.

16). The prefilled syringe assembly of claim 1 further comprising a means of releasably engaging an injection device;

17). The prefilled syringe assembly of claim 1 further comprising a locking mechanism disposed on the syringe barrel for interfacing with an injection device;

18). The prefilled syringe assembly of claim 1 further comprising at least one locking tab disposed at the proximal end of the syringe barrel for interfacing with an injection device;

19). The prefilled syringe assembly of claim 18 wherein the at least one locking tab further comprises:
(a) a first pair of locking tabs positioned 180° opposite a second pair of locking tabs disposed on an outer surface of the syringe barrel and about the longitudinal axis; and
(b) a third locking tab positioned 180° opposite a fourth locking tab disposed on the outer surface of the syringe barrel and about the longitudinal axis, wherein the first and second pairs of locking tabs are 90° offset from the third and fourth locking tabs.

20). The prefilled syringe assembly of claim 18 further comprising a flat ring extending away from the longitudinal axis and disposed at the proximal end of the syringe barrel serving as a mechanical locating device for positioning the syringe assembly at a filling station.

21). A prefilled syringe assembly designed for injecting a medical fluid into a site, the assembly comprising:
(A) a syringe barrel of cylindrical configuration having a longitudinal axis extending between a distal end and a proximal end, the syringe barrel including:
(1) an inner surface defining a cylindrical chamber having said medical fluid therein;
(2) a shoulder portion connected to the cylindrical chamber;
(3) a neck portion connected to the shoulder portion;
(4) a tapered tip terminating at the distal end, the tapered tip extending a first distance along the longitudinal axis from the neck portion toward the distal end and having:
(a) a shaft; and
(b) a bore therethrough; and
(5) a luer collar terminating at the distal end and extending a second distance along the longitudinal axis from the neck portion toward the distal end such that the second distance is generally equal to the first distance, the luer collar including;
(a) a base portion connecting the luer collar to the tapered tip;
(b) an outer wall connected to the base portion and the neck portion, the outer wall protecting the tapered tip during handling; and
(B) a piston received through the proximal end and positioned within the syringe barrel, the piston adapted for movement along the longitudinal axis.
(C) an elastomeric plug of cylindrical configuration, the plug disposed at the distal end and including:
(1) a widened top portion facilitating the removal thereof;
(2) an outer skirt connected to the widened top portion and contiguous with the outer wall of the luer collar; and
(3) an inner skirt connected to the widened top portion and contiguous with the shaft of the tapered tip.

22). The prefilled syringe assembly of claim 21 wherein the elastomeric plug minimizes microscopic crazing of the tapered tip and/or luer collar during steam sterilization and protects the tapered tip and luer collar from breakage during handling.

23). The prefilled syringe assembly of claim 21 wherein the elastomeric plug is made of a material selected from the group consisting of natural rubber, butyl or halobutyl rubber.

24). The prefilled syringe assembly of claim 21 wherein the plug may be made from a plastic, an elastomeric compound or a combination of plastic and elastomeric compound.

25). The prefilled syringe assembly of claim 21 further comprising a means of releasably engaging an injection device;

26). The prefilled syringe assembly of claim 21 further comprising a locking mechanism disposed on the syringe barrel for interfacing with an injection device;

27). The prefilled syringe assembly of claim 21 further comprising at least one locking tab disposed at the proximal end of the syringe barrel for interfacing with an injection device;

28). The prefilled syringe assembly of claim 26 wherein the at least one locking tab further comprises:
(a) a first pair of locking tabs positioned 180° opposite a second pair of locking tabs disposed on an outer surface of the syringe barrel and about the longitudinal axis; and
(b) a third locking tab positioned 180° opposite a fourth locking tab disposed on the outer surface of the syringe barrel and about the longitudinal axis, wherein the first and second pairs of locking tabs are 90° offset from the third and fourth locking tabs.

29). The prefilled syringe assembly of claim 26 further comprising a flat ring extending away from the longitudinal axis and disposed at the proximal end of the syringe barrel for preventing medical fluid from dripping into the injection device.

30). The prefilled syringe assembly as in any one of claims 1, 8, 11 and 21 in which the prefilled syringe assembly may be used as a hand operated device or in conjunction with a semi-automatic injection device or in conjunction with an automatic injection device.
